(19) United States
(12) Patent Application Publication
(10) Pub. No.: US 2024/0156716 A1
(43) Pub. Date: May 16, 2024

(54) COSMETIC USE OF A SOLANUM LYCOPERSICUM FRUIT (TOMATO) SKIN EXTRACT

(71) Applicant: INATURALS, AVIGNON (FR)

(72) Inventors: LEILA FALCAO, AVIGNON (FR); NICOLAS ISSALY, VALENCIA A CORUÑA (ES)

(21) Appl. No.: 18/575,342

(22) PCT Filed: Jun. 30, 2022
(86) PCT No.: PCT/EP2022/068203

§ 371 Date: Dec. 29, 2023

(30) Foreign Application Priority Data
Jul. 1, 2021 (EP) 21305913.2

(51) Int. Cl. A61K 8/9789; A61K 8/31; A61K 8/34; A61K 8/63; A61Q 17/00; A61Q 19/00; A61Q 19/08; A61Q 19/10

(52) U.S. Cl. CPC A61K 8/9789; A61K 8/31; A61K 8/34; A61K 8/63; A61Q 17/005; A61Q 19/007; A61Q 19/08; A61Q 19/10

(57) ABSTRACT

The present invention relates to the cosmetic use of a lipophilic Solanum lycopersicum fruit peel extract comprising at least 5% by weight of amyrins, at least 1.5% by weight of sterols, and at most 0.5% by weight of lycopene for maintaining, restoring and/or balancing, or reinforcing the natural defenses of the skin or mucous membranes.

### FIELD OF THE INVENTION

The present invention relates to the cosmetic use of a Solanum lycopersicum fruit (Tomato) peel extract as an active ingredient for maintaining, restoring and/or balancing, or reinforcing the natural defenses of the skin or mucous membranes.

### TECHNICAL BACKGROUND

The barrier of the skin is composed of a physical barrier and a chemical barrier both involved in the natural defenses system of the skin.

Epidermis, the top epithelial layer of the skin, is composed of keratinocytes which undergo proliferation and differentiation processes resulting in constant renewal of the upper skin layers. Epidermal keratinocytes play an important role as a barrier against diverse environmental factors. Specifically, the keratinocyte differentiation programme lead to the formation of dead cells (corneocytes) in the uppermost skin layer. The cornified skin layer provides many advantages to the face and tissues, including an increase in elasticity, stability, moisturizing, and mechanical resistance.

A wide range of specialized cells, keratinocytes, melanocytes, tissue resident leukocytes (dendritic cells) and soluble mediators like antimicrobial peptides (AMP), or lipid mediators (cytokines) contribute to the epidermal barrier.

The skin is the first barrier against viruses, bacteria, fungi, parasites and toxins. Innate immune defense against pathogens occurs by physical barriers, by recruitment of cells such as neutrophils, NK cells, and macrophages, and by secretion of antimicrobial peptides. Three types of epidermal cells participate in the immune response: keratinocytes, melanocytes and Langerhans cells. These cells, which are found only in the skin, play a key role innate and adaptative systems. Notably, keratinocyte produce chemokines for the recruitment and activation of leukocytes (innate immune response) and are antigen-presenting cells leading to the acquired immune system activation. Langerhans cells, which are found in the top layer of the epidermis, detect foreign elements and alert other cells to activate immune functions.

Any cell depletion, poor immune regulation or functional deficit is likely to favour the occurrence of manifestations including non-pathological alterations of skin visual aspect and/or mechanical properties, cutaneous discomfort, cutaneous pathological disorders, and greater sensitivity to microbial aggression. Cutaneous immune disorders are normal physiological phenomena that appear with age but that can be accelerated by microorganism infections (viruses and bacteria), physical or chemical stresses, chronological ageing, ultraviolet rays, urban living conditions, etc. The accumulation of factors such as stress, poor diet, environmental, chemical or physical aggressors including sunlight, pollution, dryness or heat can diminish the capabilities of these protective cells.

Antimicrobial peptides (AMPs), also called host defence peptides (HDPs) are part of the innate immune response found among all classes of life. There are numerous AMPs identified in keratinocytes, including psoriasin (S100A7), koebnerisin (S100A7A), lactotransferrin (LTF), Calcium binding protein (S100B), human β-defensins (HBD) and cathelicidin (LL-37). In addition to their antimicrobial effects, they have roles in migration of granulocytes, dendritic cells, and T lymphocytes and the activation of both innate and acquired immune responses.

The stimulation of antimicrobial peptides in keratinocytes would make it possible to enhance and/or to restore the natural defenses in the healthy or diseased skin. This stimulation would thus make it possible to advantageously supplement the skin's passive defence system as made up by the stratum corneum (corneocytes+intercellular cement), and to prepare the adaptive immune response in newborn infants, children, adults, and aged persons, whether in good health or not.

An active ingredient acting on these AMPs production would thus enhance the natural defenses of the skin and in particular the skin innate immune response, typically to fight physical, chemical and microbial insults.

There is thus a need for a novel active ingredient that stimulates the production of antimicrobial peptides in keratinocyte.

### SUMMARY OF THE INVENTION

The invention relates to the cosmetic use of a lipophilic Solanum lycopersicum fruit peel extract for maintaining, balancing, or reinforcing the natural defenses of the skin or mucous membranes.
In some embodiments, the lipophilic Solanum lycopersicum fruit peel extract is depleted in lycopene.
The cosmetic use of the invention is preferably for reinforcing skin barrier function, and/or maintaining or increasing the resistance or the tolerance of the skin against an external aggression or stress such as chemical, physical or microbial aggression, and/or preventing or treating a non-pathological alteration of the skin caused by an aggression or stress such as chemical, physical or microbial aggression.
In some embodiments, the lipophilic Solanum lycopersicum fruit peel extract is used as a soothing agent, in particular for improving or restoring skin comfort and/or for restoring and/or maintaining a healthy skin microbiome.
In a particular embodiment, the lipophilic Solanum lycopersicum fruit peel extract is preferably used in combination with a vegetable or mineral oil, preferably a vegetable oil. The weight ratio of the lipophilic Solanum lycopersicum fruit peel extract to the vegetal or mineral oil is preferably from 1:2 to 1:70. The vegetal oil and the lipophilic Solanum lycopersicum fruit peel extract may be obtained by simultaneous or separate extraction(s) of tomato byproducts, e.g. by supercritical CO2 extraction.
According to the invention, the lipophilic Solanum lycopersicum fruit peel extract advantageously comprises at most 0.5%, preferably at most 0.3% by weight, preferably at most 0.2% by weight, preferably at most 0.1% by weight, of lycopene, in relation to the total weight of the extract.
The lipophilic Solanum lycopersicum fruit peel extract preferably comprises at least 5% by weight of amyrins, in particular of α-amyrin, β-amyrin and/or δ-amyrin, in relation to the total weight of the extract, more preferably the lipophilic Solanum lycopersicum fruit peel extract comprises at least 1.5% by weight of α-amyrin, at least 1% by weight of β-amyrin and at least 2.5% by weight of δ-amyrin, in relation to the total weight of the extract.
The lipophilic Solanum lycopersicum fruit peel extract advantageously further comprises at least 1.5% by weight of sterols, in relation to the total weight of the extract, and preferably at least 1% by weight of β-sitosterol and stigmasterol, in relation to the total weight of the extract.
In some embodiments, the lipophilic Solanum lycopersicum fruit peel extract is present in a cosmetic composition for topical administration in a weight content from 0.01% to 30%, in particular from 0.1% to 5% in relation to the total weight of the composition, said composition comprising at least one cosmetic acceptable excipient.
In some embodiments, the lipophilic Solanum lycopersicum fruit peel extract, the combination of the lipophilic Solanum lycopersicum fruit peel extract with the vegetable oil, or the cosmetic composition, is topically applied or orally administered to a healthy subject having a sensitive skin, a dry skin, an aged-skin, a blemish-prone skin, an acne-prone skin, an atopic-prone or a skin weakened by an external insult or stress.
The invention also relates to an oral or topical composition comprising a lipophilic Solanum lycopersicum fruit peel extract depleted in lycopene, as an active ingredient, preferably as defined above, for use in treating an acne-prone skin, blemish-prone skin and/or an atopic-prone skin.
The invention also relates to an oral or topical composition comprising a lipophilic Solanum lycopersicum fruit peel extract depleted in lycopene, as an active ingredient, preferably as defined above, for use in treating a skin disease characterized by an altered or unbalanced innate immune response, preferably selected from eczema, psoriasis, rosacea, acne, dermatitis, atopic dermatitis, and irritative skin.
The invention also relates to a lipophilic Solanum lycopersicum fruit peel extract comprising:
at most 0.3% by weight, preferably at most 0.2% by weight, preferably at most 0.1% by weight, of lycopene, in relation to the total weight of the extract;
at least 5% by weight of amyrins, in particular of α-amyrin, β-amyrin and/or δ-amyrin, in relation to the total weight of the extract;
at least 1.5% by weight of sterols, in relation to the total weight of the extract; and preferably at least 1% by weight of β-sitosterol and stigmasterol, in relation to the total weight of the extract.
   The invention also relates to cosmetic composition comprising a lipophilic Solanum lycopersicum fruit peel extract as defined above, preferably in a weight content from 0.01% to 30% in relation to the total weight of the composition, and at least one cosmetic acceptable excipient.

### BRIEF DESCRIPTION OF THE FIGURES

FIG. 1 shows the evolution of erythema levels over time depending on the tested products: a placebo composition or the extract of the invention at 1% or 2%.

FIG. 2 shows the evolution of hydration over time depending on the tested products: a placebo composition or the extract of the invention at 1% (FIG. 2A) or 0.5% (FIG. 2B).

### DETAILED DESCRIPTION OF THE INVENTION

The inventors have found that a lipophilic Solanum lycopersicum fruit peel extract may be used to enhance the skin natural defenses, in particular the skin innate immunity, e.g. through antimicrobial peptides production.

As evidenced in the experimental section, the lipophilic Solanum lycopersicum fruit peel extract used according to the invention provides transcriptional modulation, e.g. modulates the gene expression profile on ex vivo human skin explants.

Of note, the lipophilic Solanum lycopersicum fruit peel extract of the invention upregulates the expression of genes encoding antimicrobial peptide (AMPs) such as psoriasin (S100A7), koebnerisin (S100A7A), lactotransferrin (LTF), and Calcium binding protein (S100B). This upregulation of AMP genes was observed in both basal and stimulated oxidative conditions (i.e., in the presence of phorbol 12-Myristate 13-Acetate).

Thus, it is expected that the lipophilic Solanum lycopersicum fruit peel extract would stimulate innate immunity in the skin in vivo and consequently strengthen skin protective barrier. The Solanum lycopersicum fruit peel extract used in the experimental section is typically depleted in lycopene but contains significant amounts of amyrins, in particular of δ-amyrin, and sterols.

Of note, amyrins are a class of pentacyclic triterpenes ubiquitously distributed throughout the plant kingdom. α-amyrin (ursane skeleton), β-amyrin (oleanane skeleton) and δ-amyrin are the three closely related natural chemical compounds of the triterpene. ABAM (α and β-amyrins) have been shown to exhibit various pharmacological activities such as gastroprotective (Oliveira FA et al., Planta Med 2004; 70: 780-782), and hepatoprotective activities (Oliveira FA et al., J Ethnopharmacol 2005; 98: 103-108). In contrast, δ-amyrin is not ubiquitously distributed among plants.

To the knowledge of the inventors, the prior art does not describe that Solanum lycopersicum fruit extracts rich in amyrins and depleted in lycopene would promote skin innate immunity, in particular by inducing the expression of AMPs.

Thus, the invention relates to the cosmetic, non-therapeutic, use of a Solanum lycopersicum fruit peel extract, for maintaining, balancing or reinforcing the natural defenses of the skin or mucous membranes.

In the context of the invention, “Solanum lycopersicum fruit” or “tomato” refers to the edible berry of the plant Solanum lycopersicum, commonly known as a tomato plant. The terms “tomato” and “Solanum lycopersicum fruit” can be used interchangeably.

In the context of the invention, the term “Solanum lycopersicum fruit peel” refers to the outer protective layer of a tomato. The Solanum lycopersicum fruit peel comprises an epidermis covered with a cuticle (cuticular membrane), and a multi-layered hypodermis. The terms “Solanum lycopersicum fruit skin”, “Solanum lycopersicum fruit peel”, “tomato fruit peel” and “tomato fruit skin” can be used interchangeably. The terms “tomato peel extract” or “Solanum lycopersicum fruit peel extract” thus refers to a lipophilic extract of tomato peels as defined herein.

In the context of the invention, the term “skin” refers to any part of the skin of the human body, in particular the skin of the face, including the lips and eyelids, the neck, the scalp, and the skin of the hands. The term “skin” may also encompass skin appendages such as hairs and nails.

The natural defence system of the skin protects the skin against the chemical, physical or microbial aggressions and has two main aims: containing the aggression and alerting the rest of the immune system of the aggression. By maintaining, balancing (or normalizing) or reinforcing the natural defenses of the skin or mucous membranes, the skin and the skin microbiome are maintained in a good health. The skin is therefore fully able to defend against the chemical, physical or microbial aggressions while maintaining its visual aspect and its mechanical properties.

In the context of the invention, the term “aggression” or “stress” refers to any exposure of the skin to an external condition which may alter or trigger the immune response of the skin. These terms may also refers to internal or emotional stress which may also alter or trigger the immune response of the skin.

In the context of the invention, the chemical aggressions include aggression by allergens, pollutants (e.g. exhaust gases, fine particles, volatile organic compounds), tabaco, irritant agents such as organic solvents, drying agents, acidic or basic solutions, and certain therapeutic agents such as disinfectant, hydroalcoholic solutions or gels or anti-acne agent.

In the context of the invention, the physical aggressions include thermal aggression, mechanical aggression (e.g. peeling, dermo-abrasion, shaving, hair removal, laser) and electrical aggression. In a particular embodiment, the physical aggression refers to weathering conditions such as wind, cold, or heat.

In the context of the invention, the microbial aggressions include aggressions by microbiological agents such as bacteria, fungi, virus and other pathogen agents.

In certain embodiments, the aggression further encompass stress caused by the way of life such as tabaco consumption, alcohol consumption, high-fat or poor diet, consumption of ultra-processed food, emotional stress and the like.

In a particular embodiment, the aggression does not encompass aggression by UV-radiation.

In particular, the Solanum lycopersicum fruit peel extract used according to the invention enhances the production of antimicrobial peptides by skin keratinocytes and thus enhances the innate immune response of the skin.

The invention may thus be related to the cosmetic use of a Solanum lycopersicum fruit peel extract, for maintaining, balancing or reinforcing the innate immune response of the skin or mucous membrane. The invention may also be related to the cosmetic use of a Solanum lycopersicum fruit peel extract, for contributing to normal functioning of the immune system, in particular the skin immune system, and/or for boosting the skin innate immunity. The cosmetic use of the Solanum lycopersicum fruit peel extract may also be for restoring an immune imbalance of the skin.

In a particular embodiment, the Solanum lycopersicum fruit peel extract is thus used in the cosmetic field for reinforcing skin barrier function and/or maintaining or increasing the resistance or the tolerance of the skin against an external aggression or stress such as chemical, physical or microbial aggressions.

In the context of the cosmetic uses of the invention, the Solanum lycopersicum fruit peel extract may be topically applied on a healthy area of the skin or orally administered to a subject having healthy skin.

As used herein, a “healthy skin” or “a healthy skin area” refers to skin which is not afflicted with any cutaneous disease or wound. But the term “healthy skin” also encompasses skin showing a non-pathological, altered, immune response to external aggression or stress and/or non-pathological alteration of the visual aspect or the mechanical properties of the skin as described further below.

In some embodiments, the Solanum lycopersicum fruit peel extract is administered to a subject having a sensitive skin, a dry skin, an aged-skin, a blemish-prone skin, an acne-prone skin, an atopic-prone or a skin weakened by an external aggression or stress such as chemical, physical or microbial aggressions.

By increasing the resistance of the skin to external aggressions, the Solanum lycopersicum fruit peel extract can prevent or alleviate non-pathological alterations of the skin caused by an exposure to an external aggression.

Accordingly, the invention also relates to the use of the Solanum lycopersicum fruit peel extract to prevent or treat a non-pathological alteration of the skin caused by an aggression or stress, e.g. as described above.

As used herein, a non-pathological alteration of the skin refers to non-pathological modification of the appearance or the mechanical properties of the skin. Non-pathological alterations of the skin encompass without being limited to a thinning of the skin, in particular of the epidermis, a loss of radiance of the skin, dark circles, a dull complexion, a loss of skin elasticity, an alteration of the smooth aspect of the skin, an increase in the roughness of the skin redness, dryness and the like.

Within the scope of the present invention, by “preventing a non-pathological alteration” is meant the fact of preventing, slowing down or delaying the occurrence of said skin alteration.

By “treating a non-pathological alteration” is meant the fact of correcting, attenuating, diminishing, making less visible, reducing the appearance or even making the skin alteration disappear.

In further embodiments, the invention relates to the cosmetic use of a Solanum lycopersicum fruit peel extract, preferably depleted in lycopene, for maintaining, restoring or balancing skin microbiota.

As used herein, the terms “skin microbiota”, “skin microbiome” or “skin flora” refer to the microorganisms which reside on the skin, typically human skin and encompass bacteria, mycobacteria, viruses, fungi and parasitic germs. Most are found in the superficial layers of the epidermis and the upper parts of hair follicles. Skin flora is usually non-pathogenic, and either commensal or mutualistic. The benefits that such microorganisms can offer include preventing transient pathogenic organisms from colonizing the skin surface, either by competing for nutrients, secreting chemicals against them, or stimulating the skin's immune system.

By “restoring” or “balancing” or “normalizing” skin microbiota, it is meant restoring microorganism's diversity so as to get a balance in microorganism distribution promoting skin health and/or corresponding to healthy skin. For instance, the Solanum lycopersicum fruit peel extract may enable to decrease the abundance of bacterial strains which may be involved in blemishes or acne, e.g. such as certain C. acnes strains. In that context, the Solanum lycopersicum fruit peel extract can be typically used in subjects having acne-prone skin or blemish-prone skin.

The Inventors further showed that the Solanum lycopersicum fruit peel extract modulates the expression of genes involved in inflammation, and oxidative stress in both basal and stimulated conditions.

Indeed, the Solanum lycopersicum fruit peel extract induced a strong downregulation of genes involved in inflammation (mainly for CXCL8, IL6, CXCL2, CXCL3, CCL20, TNFAIP3, TNFAIP6), and in oxidative stress (PTGS2, GPX3, HMOX1), and in terminal keratinocyte differentiation (KRT17, SPRR1A, TGM1 , several LCE, EREG, CDSN and IVL).

Such downregulation may also contribute to maintain and reinforce a normal skin barrier function, e.g. by preventing overreaction of the skin to external insults or stress.

Besides the Solanum lycopersicum fruit peel extract according to the invention can further be used to reduce inflammation when skin is under oxidative stress and production of

Reactive Oxygen Species. More generally, the Solanum lycopersicum fruit peel extract can exhibit a soothing effect, e.g. via the inhibition of the oxidative stress and an anti-inflammatory effect.

In a further embodiment, the Solanum lycopersicum fruit peel extract is thus used as a soothing agent, in particular for improving or restoring skin comfort as well as for preventing or alleviating skin discomfort.

As used herein, the term “skin discomfort” refers to a set of unpleasant sensations, not linked to a pathology, such as sensations of tightness, tingling, itching or heating, sensations of dry or dehydrated skin, and transient redness. These sensations are not linked to a pathology and can be induced by certain external factors (for example prolonged exposure to the sun, wind or cold), by certain cosmetic treatments (for example by too frequent washing with water limestone or with a hydroalcoholic solution, aggressive cosmetic treatments such as peeling, shaving, exfoliation, hair removal, coloring, permanent) or be associated with so-called sensitive, hypersensitive, reactive, intolerant or even atopic-prone skin.

For instance, the Solanum lycopersicum fruit peel extract can be used to alleviate, and more generally to manage sensitive, reactive or intolerant skin.

The term “sensitive, reactive or intolerant skin” refers to a skin characterized by a disproportionate response to external factors which causes skin discomfort. This increased reactivity can result from an alteration, non-pathological, of the barrier function of the skin, and more generally of the skin innate immune system, which decreases the tolerance threshold of the skin to external stimuli.

More particularly, the Solanum lycopersicum fruit peel extract may be also used as a soothing agent on the skin by controlling skin response to oxidative stress and/or by controlling inflammatory skin response.

In a more general aspect, the invention may also be related to a method for the cosmetic treatment of sensitive skin, dry skin, dehydrated skin, intolerant skin, aged-skin, a blemish-prone skin, an acne-prone skin, an atopic-prone skin, a skin weakened by an external aggression or stress such as chemical, physical or microbial aggressions, a skin which present a non-pathological immunological imbalance or non-pathological reddening, characterised in that it implies applying onto the skin and/or the mucous membrane or orally administering a Solanum lycopersicum fruit peel extract as defined herein.

The invention may also be related to a cosmetic care method for skin and/or mucous membranes, in view of improving their condition and/or appearance, consisting of topically applying or orally administering to a subject a Solanum lycopersicum fruit peel extract as defined herein.

In the context of the invention, the terms “improving the condition and/or appearance of the skin” include:
Unifying skin tone;
Balancing and/or restoring a more luminous and/or brighter and/or clearer skin tone;
Balancing and/or restoring skin firmness and/or promoting firming and antiaging of the epidermis, especially of the face skin.
Balancing and/or moisturizing capacity of skin, especially the face skin;
Preventing or treating a dull skin tone and/or a blurred skin tone;
Preventing or treating skin pigmentation irregularities, including pigmentation spots induced by an external aggression or stress;
To make the skin, especially on the face, fresher and more radiant;
Soothing the skin's appearance.
Promoting or accelerating the recovery of the epidermis, especially on the face;
Revitalizing the skin, especially on the face;
Increasing the epidermis thickness;
Balancing and/or promoting a healthy skin tone; and/or
Preventing, treating or restoring the skin's barrier function.

In a further embodiment, the Solanum lycopersicum fruit peel extract is used as a immunity skin boost agent and/or a skin protective agent by controlling skin response to oxidative stress and enhancing antimicrobial peptides production by skin keratinocytes.

In the above described cosmetic methods and uses, the Solanum lycopersicum fruit peel extract is preferably used as a cosmetic agent, i.e. as an active agent having a cosmetic effect. By “active agent with a cosmetic effect” it is meant a compound having an action on the skin resulting in at least one cosmetic effect on the skin. By “cosmetic effect” is meant any non-therapeutic effect aiming at modifying and/or improving the aspect, the mechanical properties or the feeling of the skin or the mucosa, and/or protecting them from non-pathological modification of the skin caused by an external aggression.

The invention also relates to an oral or topical composition comprising a lipophilic Solanum lycopersicum fruit peel extract depleted in lycopene, as an active ingredient, preferably as defined above, for use in treating an acne-prone skin, blemish-prone skin and/or an atopic-prone skin.

The invention further relates to an oral or topical composition comprising a Solanum lycopersicum fruit peel extract, advantageously depleted in lycopene, preferably as defined herein, as an active ingredient, for use for treating a skin disease characterized by an altered or unbalanced innate immune system, preferably selected from eczema, psoriasis, rosacea, acne, dermatitis, atopic dermatitis, and irritative skin.

The invention further relates to the use of a Solanum lycopersicum fruit peel extract, advantageously depleted in lycopene, preferably as defined herein, to produce a pharmaceutical or dermatological composition for treating a skin disease characterized by an altered or unbalanced innate immune system, preferably selected from eczema, psoriasis, rosacea, acne, dermatitis, atopic dermatitis, and irritative skin.

The invention further relates to a method for treating a skin disease characterized by an altered or unbalanced innate immune system, preferably selected from eczema, psoriasis, rosacea, acne, dermatitis, atopic dermatitis, and irritative skin, comprising the administration, in particular topical or oral administration, of an effective amount of a Solanum lycopersicum fruit peel extract, advantageously depleted in lycopene, preferably as defined herein, to a subject in need thereof.

In a further embodiment, the Solanum lycopersicum fruit peel extract as defined herein may be for use for preventing or treating disorders or diseases of the skin and/or mucous membranes, whether immature, normal or mature/elderly.

In a further embodiment, the invention may relate to the use of Solanum lycopersicum fruit peel extract as defined herein, to produce a pharmaceutical or dermatological composition for preventing or treating disorders or diseases of the skin and/or mucous membranes, whether immature, normal or mature/elderly.

In a further embodiment, the invention may relate to a method for preventing or treating disorders or diseases of the skin and/or mucous membranes, whether immature, normal or mature/elderly, comprising the administration, in particular topical or oral administration, of an effective amount of a Solanum lycopersicum fruit peel extract, advantageously depleted in lycopene, preferably as defined herein, to a subject in need thereof.

In the above embodiments, the disorders or diseases of the skin and/or mucous membranes may be selected in the group consisting of the inflammatory diseases, oxidation diseases, blotchiness, disorders related to radical attacks linked to chemical or atmospheric pollution and/or linked to exposure to UV or IR radiation, barrier or homeostasis disorders, aging, in particular chronological aging and/or actinic aging, and/or physical, chemical or microbial aggressive factors, more advantageously inflammatory and irritative diseases, or barrier or homeostasis disorders.

Advantageously, the inflammatory or irritative diseases of the skin, or the barrier or homeostasis disorders of the skin are: acne, rosacea or erythrocouperose, vascular disorders, in particular blotchiness, seat dermatitis, atopic dermatitis, eczema, contact dermatitis, irritative dermatitis, allergic dermatitis, seborrheic dermatitis (cradle cap), xerosis, cutaneous erythema, elderly or photo-aged skin, photosensitive skin, pigmented skin (melasma, post inflammatory pigmentation, etc.), skin with stretch marks, sunburn, irritation by chemical, physical, bacteriological and fungal agents, and disorders related to radical attacks linked to chemical or atmospheric pollution and/or linked to exposure to UV or IR radiation.

Combination of the Solanum lycopersicum Fruit Peel Extract and a Vegetable Oil

In an advantageous embodiment of the invention, the Solanum lycopersicum fruit peel extract is used in combination with a lipophilic vehicle, preferably a vegetable or mineral oil, more preferably a vegetable oil. In particular, the Solanum lycopersicum fruit peel extract is diluted into the vegetable oil.

The inventors found that by combining the Solanum lycopersicum fruit peel extract used according to the invention with a lipophilic vehicle such as a vegetable oil, the compounds of particular interest for the uses according to the invention, including the amyrins and the sterols, have a greater bioaccessibility. The cosmetic, dermatological or pharmaceutical activity of the Solanum lycopersicum fruit peel extract is thus enhanced when said extract is diluted into the lipophilic vehicle such as the vegetable oil.

Advantageously, the lipophilic vehicle is a vegetable oil.

Advantageously, the vegetable oil used according to the invention is an oil selected in the group consisting of tomato seed oil, apple seed oil, pear seed oil, sunflower oil, palm oil, palm kernel oil, coconut oil, grapeseed oil, black mustard oil, poppyseed oil, karite butter oil, sweet almond oil, soybean oil, avocado oil, groundnut oil, cotton oil, sesame oil, olive oil, corn oil, cocoa bean oil, castor oil, behen oil, flax oil, rapeseed oil, annatto oil, wheatgerm oil, safflower oil, walnut oil, hazelnut oil and turnip seed oil. Preferably, the vegetable oil used according to the invention is a vegetable seed oil and more preferably a tomato seed oil.

In a particular embodiment, the weight ratio of the Solanum lycopersicum fruit peel extract to the vegetable or mineral oil, preferably the vegetable oil, is from 1:70 to 1:1, such as 1:50 to 9:10 for instance from 0.1 to 0.9, e.g. from 0.2 to 0.8 or from 0.3 to 0.6 such as 1:2 (0.5).

For instance, the Solanum lycopersicum fruit peel extract can be combined with the lipophilic vehicle, preferably the tomato seed oil, so as to give an intermediate composition with a weight ratio of the Solanum lycopersicum fruit peel extract to the lipophilic vehicle from 0.1 to 0.9 e.g. about 0.5. This intermediate composition is preferably incorporated in a cosmetic composition to be applied on the skin. For instance, such an intermediate composition may account for 0.1% to 10% by weight of the total weight of the cosmetic composition to be applied on the skin.

As another example, the Solanum lycopersicum fruit peel extract can be diluted in the lipophilic vehicle according to a weight ratio of the Solanum lycopersicum fruit peel extract to the vegetable or mineral oil ranging from 1:75 to 1:20, in particular, from 1:60 to 1:30, preferably from 1:55 to 1:45 such as about 1:49. In such an embodiment, the combination may be directly applied to the skin or administered by oral route.

In a particular embodiment, the vegetable oil used according to the invention is a tomato seed oil that preferably comprises at least 40% by weight, in particular from 45% to 70% by weight, of linoleic acid, in relation to the total weight of the tomato seed oil. The tomato seed oil used in the invention may further comprise at least 10% by weight, in particular from 10% to 30% by weight, of oleic acid, in relation to the total weight of the tomato seed oil. The tomato seed oil used in the invention may further comprise at least 5% by weight, in particular from 5% to 15% by weight, of oleic acid, in relation to the total weight of the tomato seed oil. For instance, the vegetal oil may comprise from 45% to 55% by weight of linoleic acid, from 15% to 25% by weight of oleic acid and from 5% to 15% by weight of palmitic acid.

Composition Comprising the Solanum lycopersicum Fruit Peel Extract or the Combination of Said Extract with a Vegetable Oil

The Solanum lycopersicum fruit peel extract is typically administered topically or orally in the form of a composition.

According to the invention, the Solanum lycopersicum fruit peel extract may be used alone as such, in combination with a vegetable or mineral oil, or may be present as an active ingredient in a cosmetic, dermo-cosmetic, pharmaceutical or a nutraceutical composition such as a dietary supplement, preferably in a cosmetic, dermo-cosmetic, or a nutraceutical composition. When the Solanum lycopersicum fruit peel extract is combined with a vegetable or mineral oil, the combination may also be present in a cosmetic, dermo-cosmetic, pharmaceutical or a nutraceutical composition such as a dietary supplement, preferably in a cosmetic, dermo-cosmetic, or a nutraceutical composition.

The composition according to the invention is preferably formulated for a topical or oral administration, in particular for a topical administration.

Advantageously, the Solanum lycopersicum fruit peel extract is present in the composition in a weight content from 0.001% to 20%, 0.01% to 10%, in particular from 0.1% to 5% in relation to the total weight of the composition, said composition comprising at least one excipient.

Depending on its type (cosmetic, pharmaceutical or dermatological), the composition according to the invention further include at least one cosmetically, pharmaceutically, nutraceutically or dermatologically acceptable excipient. In particular, the composition according to this invention can further include at least one excipient that is cosmetically, pharmaceutically, nutraceutically or dermatologically known to the person skilled in the art, chosen from amongst surfactants and/or emulsifiers, thickeners, preservatives, chemical or mineral filters, hydrating agents, buffering agents, chelating agents, denaturants, opacifying agents, pH adjusters, reducing agents, stabilizing agents, thermal waters, gelling agents, film-forming polymers, fillers, mattifying agents, gloss agents, pigments, dyes, perfumes, and mixtures thereof. The person skilled in the art knows how to adapt formulation of the composition according to the invention by using its general knowledge. The CTFA (Cosmetic Ingredient Handbook, sixteenth Edition (2016)) also describes various cosmetic excipients suitable for use in the present invention.

The composition according to the invention can be formulated in the form of different preparations adapted to topical administration and includes creams, emulsions, microemulsion, nanoemulsion, serum, soap, gel, milks, ointments, lotions, oils, aqueous, alcoholic or glycolic solutions, powders, patches, sprays, shampoos, varnishes, foam or any other product for external application.

Indeed, the composition according to the invention may be in the form of a cosmetic product of any type such as a cosmetic care product, a makeup product or a body hygiene product.

The composition according to the invention can also be formulated in the form of different preparations adapted to oral administration and includes tablets, capsules, coated tablets, syrups, suspensions, solutions, powders, pellets, emulsions, suspensions of microspheres or nanospheres, lipid vesicle suspensions or various polymer-based vesicles.

Advantageously, the composition is a topical cosmetic composition.

The cosmetic composition may also comprise other cosmetic active ingredients. Many cosmetically active ingredients are known to those skilled in the art to improve the health and/or the physical appearance of the skin. On the other hand, the compounds described in the present invention can have a synergistic effect when combined with each other. These combinations are also covered by the present invention. The CTFA Cosmetic Ingredient Handbook, sixteeth Edition (2016) describes various cosmetic and pharmaceutical ingredients commonly used in the cosmetic and pharmaceutical industry, which are particularly suitable for topical use. Examples of these classes of ingredients include, but are not limited to, the following compounds: abrasive, absorbents, cosmetic compound such as perfumes, pigments, dyes, essential oils, astringents, for example olive oil. clove, menthol, camphor, eucalyptus oil, eugenol, menthyl lactate, witch hazel distillate, anti-acne agents, anti-flocculants, anti-foam agents, antimicrobial agents (for example: iodopropyl butylcarbamate), antioxidants, binders, biological additives, buffering agents, blowing agents, chelating agents, additives, biocidal agents, denaturants, thickeners, and vitamins, and derivatives or equivalents thereof, film-forming materials, polymers, opacifying agents, pH adjusters, reducing agents, depigmenting or brightening agents (for example: hydroquinone, kojic acid, ascorbic acid, magnesium ascorbyl phosphate, ascorbyl glucosamine), conditioning agents (for example: humectants).

Advantageously, the Solanum lycopersicum fruit peel extract used according to the invention, alone or combined with lipophilic vehicle such as a vegetable or mineral oil, is the sole active ingredient of the composition.

In a particular embodiment, the composition used according to the invention may comprise (expressed in weight content in relation to the total weight of the composition):
from 0.001% to 20% of the Solanum lycopersicum fruit peel extract, in particular 0.01 to 10%, more particularly from 0.1% to 5%;
from 60% to 99.999% of at least one excipient; and
from 0% to 20% of an additional active ingredient.

The dosages and optimum galenic formulations according to the invention can be established according to the criteria usually taken into account when formulating a pharmacological, dermatological, nutraceutical or cosmetic treatment suited to a patient or an animal, such as for example the age and body weight of the patient or animal, severity of the general condition, tolerance of treatment, side effects observed, skin type.

The Solanum lycopersicum Fruit Peel Extract and Process for Obtaining the Same

The Solanum lycopersicum fruit peel extract according to the invention is a lipophilic Solanum lycopersicum fruit peel extract obtained from the extraction of Solanum lycopersicum fruit peels, and in particular from Solanum lycopersicum fruit peels of tomato by-products from industrial or food waste. The tomato by-products from which the extract is obtained is typically tomato pomace from food industry. When the tomato by-product comprises tomato peels and seeds, the seeds and the skins may be separated, for example by screening and the skins are recovered.

The Solanum lycopersicum fruit peel extract according to the invention is a lipophilic Solanum lycopersicum fruit peel extract. The Solanum lycopersicum fruit peel extract of the invention exhibits a poor solubility in water as compared to lipidic media such as vegetable oil. The Solanum lycopersicum fruit peel extract of the invention is preferably a Solanum lycopersicum fruit peel wax, also called Solanum lycopersicum fruit (tomato) skin wax. The Solanum lycopersicum fruit peel extract of the invention is preferably depleted in lycopene and preferably comprise amyrins and sterols.

In a preferred embodiment of the invention, the Solanum lycopersicum fruit peel extract used is depleted in lycopene. Advantageously, the extract comprises at most 0.5% preferably at most 0.3% by weight, preferably at most 0.2% by weight, preferably at most 0.1% by weight, more preferably at most 0.05% by weight, of lycopene, in relation to the total weight of the extract. In particular, the Solanum lycopersicum fruit peel extract used comprises from 0% to 0.1% by weight, preferably from 0% to 0.05% by weight, of lycopene, in relation to the total weight of the extract.

Lycopene is a carotenoid. In a particular embodiment of the invention, the Solanum lycopersicum fruit peel extract is depleted in carotenoids. More particularly, the extract comprises at most 0.6%, preferably at most 0.3% by weight, in particular at most 0.2% by weight, of carotenoids, in relation to the total weight of the extract. In the context of the invention, the weight content of carotenoids and in particular of lycopene, is determined by methods known by the one skilled in the art, such as by HPLC-DAD according to the method described by Gleize et al. (Gleize, B., M. Steib, M. André, and E. Reboul. 2012. Simple and fast HPLC method for simultaneous determination of retinol, tocopherols, coenzyme Q10 and carotenoids in complex samples. Food Chemistry 134: 2560-2564). In the context of the invention, “at most X %” means from 0% to X %.

In a preferred embodiment, the Solanum lycopersicum fruit peel extract used according to the invention is rich in amyrins and in particular in δ-amyrin. In particular, the Solanum lycopersicum fruit peel extract used according to the invention comprises at least 5% by weight of amyrins, in relation to the total weight of the Solanum lycopersicum fruit peel extract. Advantageously, the Solanum lycopersicum fruit peel extract used according to the invention comprises from 5% to 32% by weight of amyrins, in relation to the total weight of the Solanum lycopersicum fruit peel extract. For example, the Solanum lycopersicum fruit peel extract used according to the invention comprises from 5% to 25% by weight, from 5% to 20% by weight, from 5% to 15% by weight, or from 5% to 10% by weight, of amyrins, in relation to the total weight of the Solanum lycopersicum fruit peel extract.

Amyrins are triterpene compounds that can be found in the form of α-amyrin, β-amyrin and δ-amyrin of the following formula (Iα), (Iβ), and (I δ):

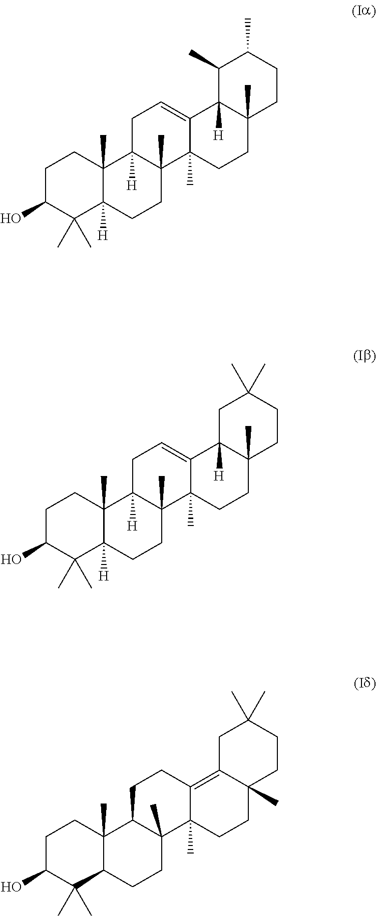

According to the invention, the Solanum lycopersicum fruit peel extract used preferably comprises α-amyrin, β-amyrin and/or δ-amyrin, more preferably the Solanum lycopersicum fruit peel extract used comprise α-amyrin, β-amyrin and δ-amyrin, in particular the Solanum lycopersicum fruit peel extract comprises at least 1.5% by weight, in particular from 1.5% to 10% by weight, of α-amyrin, at least 1% by weight, in particular from 1% to 7% by weight, of β-amyrin and at least 2.5% by weight, in particular from 2.5% to 17% by weight, of δ-amyrin, in relation to the total weight of the Solanum lycopersicum fruit peel extract. In the context of the invention, the weight content of amyrins is determined by methods known by the one skilled in the art, such as by GC-MS according to the method described by Bauer et al. (Bauer, S., E. Schulte, and H.-P. Thier. 2004. Composition of the surface wax from tomatoes: I. Identification of the components by GC/MS. European Food Research and Technology 219).

In a particular embodiment the weight ratio of δ-amyrin to the sum of α-amyrin and β-amyrin is from 0.8 to 1.2, preferably from 0.9 to 1.1.

The Solanum lycopersicum fruit peel extract used according to the invention may also be rich in sterols and in particular in β-sitosterol and stigmasterol. Preferably, the Solanum lycopersicum fruit peel extract further comprises at least 1.5% by weight, in particular from 1.5% to 12% by weight, of sterols, in relation to the total weight of the Solanum lycopersicum fruit peel extract. More preferably, the Solanum lycopersicum fruit peel extract comprises at least 1% by weight, in particular from 1% to 3% by weight, of β-sitosterol and stigmasterol, in relation to the total weight of the Solanum lycopersicum fruit peel extract. In the context of the invention, the weight content of sterols and in particular of β-sitosterol and stigmasterol, is determined by methods known by the one skilled in the art, such as by GC-MS according to the method described by Bauer et al.

The Solanum lycopersicum fruit peel extract used according to the invention may also comprise at least 10% by weight, in particular from 10% to 60% by weight, of triglycerides, in relation to the total weight of the Solanum lycopersicum fruit peel extract. In the context of the invention, the weight content of triglycerides is determined by methods known by the one skilled in the art, such as by UPLC-DAD-M.

The Solanum lycopersicum fruit (Tomato) peel extract used according to the invention may also comprise at least 10% by weight, in particular from 10% to 30% by weight, of alkanes, in relation to the total weight of the Solanum lycopersicum fruit (Tomato) peel extract. According to the invention, the sum of the contents of alkanes types C31, C32 and C33 is at least 70% by weight, in particular from 70 to 90% by weight in relation to the total alkanes content of the Solanum lycopersicum fruit (Tomato) peel extract. In the context of the invention, the weight content of alkanes is determined by methods known by the one skilled in the art, such as by GC-MS according to the method described by Bauer et al.

The Solanum lycopersicum fruit peel extract used according to the invention may also comprise at least 5% by weight, in particular from 5% to 15% by weight, of alkadienes, in particular alkadienes type C33 and C35, in relation to the total weight of the Solanum lycopersicum fruit peel extract. In the context of the invention, the weight content of alkanes is determined by methods known by the one skilled in the art, such as by GC-MS according to the method described by Bauer et al.

According to the present invention, the Solanum lycopersicum fruit peel extract may be obtained by a preparation method comprising the following steps:
a) Selecting tomato peels, in particular tomato peels from tomato pomace;
b) Optionally grinding the tomato peels
c) Extracting the tomato peels so as to obtain a lipophilic extract; and
d) Optionally filtrating the obtained extract and recovering the lipophilic tomato peel extract.

In step a), the tomato pomace obtained from industrial or food waste may comprises tomato peels and seeds. A further step of separating the tomato peels and the seeds may thus be carried out by means known by the one skilled in the art, for example by sieving (e.g. through a 1 μm mesh). The tomato peels selected in step a) may also be submitted to drying so as to obtain a residual moisture of at most 10%. The drying method are commonly known by the one skilled in the art and include heating at temperature from 40° C. to 90° C., for example in an oven or a dryer. The drying temperature should be selected in order to avoid the deterioration of the compounds of interest such as the amyrins and sterols.

In step b), the tomato peels are optionally ground by methods commonly known by the one skilled in the art and included grinding with a knife mill, cutting mill or hammer mill.

The method of preparation of the tomato peel extract may further comprises a step of removing the lycopene and advantageously the carotenoids. This step may be carried out before, during or after the extraction step c) by means commonly known by the one skilled in the art. In particular, the lycopene may be removed by physical or chemical methods known by the one skilled in the art, such as by adsorption/desorption by use of resins (i.e. macroporous adsorption resin, type column packed LX-68, HP20 or equivalent), crystallization, solvent extraction, chromatography such as countercurrent chromatography (CCC) or centrifugal partition chromatography (CPC) or other, salting out (precipitation methods), filtration methods (diafiltration, microfiltration and/or ultrafiltration or a combination thereof) thermal method, distillation or CO2 supercritical extraction. When solvent extraction methods are used for removing the lycopene, the solvent used may be selected from acetone, ethyl acetate, diethyl ether, petroleum ether, hexanes, heptanes, chloroform, and tetrahydrofuran, admixture with propylene glycol (1,2-propanediol), water and an alkali such as sodium, potassium, calcium, magnesium or ammonium hydroxide or trisodium phosphate, and mixtures thereof. Any other solvent with a Hildebrand Solubility Parameters (δ) value known between 12 and 36, in particular between 14 and 19 may also be used (A list of them can be found, for instance, in the chapter 12 “Terpene Resins” From Johannes Karl Fink, p. 303-315 of the book «Reactive Polymers Fundamentals and Applications (Second Edition)” (Plastics Design Library: 2013), 535 p.). When heating method are used for removing the lycopene, the tomato peels may be heated at temperature from 60° C. to 110° C. until removal of the lycopene, for example from 1 hour to 10 hours for example.

In step c), the extraction of the tomato peels may be carried out by several methods well known by the one skilled in the art, including physical extraction (such as hot or cold pressing on a mechanical press, or pressing on a double screw extruder), chemical extraction by means of organic solvents (such as aliphatic alkanes, alcohol, chlorinated solvents, fluorinated solvents), preferably non-polar solvent, or extraction in a supercritical medium with carbon dioxide alone and/or in combination with co-solvents, as per a vegetable oil.

In step c), when solvent extraction methods are used, the solvent may be selected from the group consisting of acetone, ethyl acetate, diethyl ether, petroleum ether, hexanes, heptanes, chloroform, and tetrahydrofuran, admixture with propylene glycol (1,2-propanediol), water and an alkali such as sodium, potassium, calcium, magnesium or ammonium hydroxide or trisodium phosphate, and mixtures thereof. The weight ratio tomato peel extract/solvent during the solvent extraction may be from 1:1 to 1:60, in particular from 1:1 to 1:20

In preferred embodiment of the invention, step c) is carried out using supercritical extraction method. The process of supercritical extraction is carried out in conventional supercritical extraction equipment that comprises of a supercritical fluid tank, a compressor, an extractor, one or more separators, a thermostatisation system and some pressure reduction valves. To carry out the supercritical extraction, the tomato peels are introduced into the extractor and the supercritical fluid, preferably CO2 alone or combined with a cosolvent such as ethanol, more preferably CO2 alone, is passed through the bed of solid starting material, under pressure and temperature conditions which permit the solubilization of the compounds of interest, in particular of the amyrins and sterols in the CO2 supercritical fluid. As the CO2 supercritical fluid crosses the bed of starting material, the CO2 supercritical fluid extracts the soluble components and then moves on to the separators, where the desired product is obtained. The CO2 supercritical fluid may be then submitted to depressurization and is then eliminated. Advantageously, the CO2 supercritical extraction step is carried out at a pressure from 200 bar to 500 bar, preferably from 200 bar to 400 bar, more preferably from 200 bar to 350 bar, and at a temperature from 40° C. to 80° C., preferably from 50° C. to 80° C., more preferably from 60° C. to 80° C. Of note, when low temperature are used (such as a temperature lower than 60° C.), higher pressure may be required (such as a pressure of at least 150 bar). Similarly, when low pressure are used (such as a pressure lower than 150 bar), high temperature should be used (such as a temperature of at least 55° C.).

In step d), the extract obtained after step c) is optionally submitted to filtration by means commonly known by the one skilled in the art, such as by sieving through a filter (e.g. a 45 μm porous filter r). This step allows the removal of the impurities. The tomato peel extract is then recovered.

In some embodiments, the process for preparing the extract according to the invention may include one or more additional steps to those mentioned above. These steps include e.g. subjecting the starting plant material to washing, freezing and/or thawing.

The process may also comprise one or more additional purification steps, preferably performed after steps c) or d) and preferably selected from a distillation step, for example molecular distillation or fractional distillation, a precipitation step, a step filtration, an extraction step, in particular liquid-liquid extraction with a suitable aqueous solution, solid-liquid on a solid support capable of trapping the compounds to be removed.

In some embodiments, the lipophilic extract obtained after step c) or d) is optionally submitted to purification step(s) for concentrating the extract in amyrins and/or sterols and/or removing certain compounds with low cosmetic effect such as alkadienes and/or alkanes . The purifications step(s) may be carried out by means commonly known by the one skilled in the art, including chromatography, specially Centrifugal Partition Chromatography (CPC) and/or molecular distillation.

In a particular embodiment of the invention, when the tomato peel extract is combined with the vegetable oil as defined herein, the vegetal oil and the tomato peel extract may be obtained by simultaneous or separate extraction(s), e.g. by physical extraction, chemical extraction or CO2 supercritical extraction. More particularly, when the tomato peel extract is combined with a tomato seed oil, said extract and said oil may be obtained by simultaneous or separate extraction(s) of tomato by-products, e.g. by physical extraction, chemical extraction or CO2 supercritical extraction, preferably CO2 supercritical extraction.

The vegetable oil used according to the invention may be obtained by means commonly known by the one skilled in the art. When a tomato seed oil is used according to the invention, the methods of preparation of said oil are similar to the one described for the tomato peel extract. In a particular embodiment, when a tomato seed oil is used according to the invention, the oil may be obtained by CO2 supercritical extraction in the following conditions: pressure from 200 bar to 450 bar, preferably from 200 to 400 bar, more preferably from 200 to 300 bar and temperature from 50° C. to 80° C., preferably from 50° C. to 70° C.

This invention will be better understood in light of the following examples, which are provided for illustrative purposes only.

### EXAMPLES

### Example 1—Preparation of a Tomato Peel Extract According to the Invention—Identification and Quantification of the Triterpenes and Carotenoids

tomato peel extract (tomato peel wax) according to the invention was prepared as follows:
a) Selecting tomato peels from tomato pomace;
b) subjecting the tomato peels to heat treatment so as to obtain a humidity lower than 10 and remove lycopene (the removal of lycopene is monitored by HPLC-DAD analysis);
c) grinding the dry mass obtained in step b) using cutting mill;
d) Extracting the tomato peels fraction by using supercritical CO2 gas at 260-300 bar and at 60-80° C.; and
e) filtering the extract obtained through 45 μm paper filter to eliminate the impurities.
   The quantification of the compounds of interest of the tomato peel extract as obtained is summarize in table 1.

TABLE 1
Identification and quantification of some
components of the tomato peel extract
Amyrins, sterols and alcanes (determined by GC-MS
according to the method described by Bauer et al.)

| | |
|---|---|
| Total amyrin | 5.3 g/100 g extract |
| α-amyrin | 1.61 g/100 g extract |
| β-amyrin | 1.07 g/100 g extract |
| δ-amyrin | 2.73 g/100 g extract |
| Stigmasterol | 0.79 g/100 g extract |
| β-sitosterol | 1.21 g/100 g extract |
| Alkanes | 14.6 g/100 g extract |
| Alkadienes | 6.82 g/100 g extract |

Carotenoids and tocopherols (determined by HPLC-DAD
according to the method described by Gleize et al.)

| | |
|---|---|
| Total carotenoids | 0.17 g/100 g extract |
| Lycopene (λmax = 476 nm) | 14.4 mg/100 g extract |
| β-caroten (λmax = 450 nm) | 30.90 mg/100 g extract |
| α-tocopherol (λmax = 347 nm) | 179.19 mg/100 g extract |

Lipids (determined by UPLC-DAD-M)

| | |
|---|---|
| Palmitic acid | 4.06 g/100 g extract |
| Steraric acid | 1.13 g/100 g extract |
| Oleic acid | 4.67 g/100 g extract |
| Linoleic acid | 17.62 g/100 g extract |
| Triglycerides | 15.34.28 g/100 g extract |
| Diglycerydes | 12.05 g/100 g extract |

Bauer et al.: Bauer, S., E. Schulte, and H.-P. Thier. 2004. Composition of the surface wax from tomatoes: I. Identification of the components by GC/MS. European Food Research and Technology 219.
Gleize et al.: Gleize, B., M. Steib, M. André, and E. Reboul. 2012. Simple and fast HPLC method for simultaneous determination of retinol, tocopherols, coenzyme Q10 and carotenoids in complex samples. Food Chemistry 134: 2560-2564.
The identification and quantification tests have been carried out in collaboration with INRAE PACA.

### Example 2—Preparation of a Combination of the Tomato Peel Extract and a Tomato Seeds Oil

A tomato seed oil was prepared as follows:
a) Selecting tomato seeds from industrial waste;
b) Drying the tomato seeds at temperature 60° C. to 110° C. until obtaining a humidity lower than 12%;
c) Grinding the dry mass obtained in step b);
d) Extracting the tomato seeds fraction by using supercritical CO2 gas:
   The separation is operated under preselected conditions: 200-300 bar and 50-70° C., so that the intended tomato seeds oil is obtained; and
   the carbon dioxide is then sent to the compressor.
e) filtering the oil obtained through 45 μm paper filter to eliminate the impurities.
   The tomato seed oil was then combined with the tomato peel extract as prepared in example 1 with a weight ratio tomato peel extract/tomato seed oil of 1/49.

### Example 3—Effects of the Tomato Peel Extract on Gene Expression in Ex Vivo Human Skin Explants Under Basal Conditions

In the present study, transcriptional effects (modulation of gene expression) of the extract as obtained in example 1, the tomato seed oil and the combination as obtained in example 2 (respectively named Tomato peel extract, tomato seed oil and combination 1/49 hereinafter) were evaluated on ex vivo human skin explants under basal conditions or under Phorbol 12-Myristate 13-Acetate (PMA)-stimulated conditions. The comparative analysis of the different transcriptomic profiles was performed using an Affymetrix GeneAtlas platform and the human “full transcriptome” U219 chip which includes 36,000 transcripts and variants.

### Materials and Methods

### Biological Models

Model: Skin explants (2 cm×2 cm) from an abdominal plastic surgery Donor E2002 (44-year-old female)
Culture conditions: 37° C., 5% CO₂

### Test Samples and Combination (Table 2)

TABLE 2

| | | | Test |
|---|---|---|---|
| Test sample | Aspect/Storage | Application | concentration |
| Tomato peel | Paste | / | Only tested in |
| extract | Storage: +4° C. | | combination |
| | protected from light | | |
| Tomato seed oil | Thick | Topical | Pure |
| | homogeneous | (5 mg/cm²) | |
| | turbid liquid | | |
| | Storage: +4° C. | | |
| | protected from light | | |
| Combination 1/49 | | Topical | 1/49 (w/v) |
| | | (5 mg/cm²) | |

### Culture and Treatment

Upon receipt of the abdominal biopsy, the adipose tissue was removed, skin explants were then cut into pieces (2 cm×2 cm) and incubated in culture medium. The test samples was then topically applied (5 mg/cm ²) or not (basal control) on the surface of the skin explant and the skin explants were pre-incubated for 24 hours. After pre-incubation:
Basal conditions
   The medium was removed and replaced with a fresh culture medium and the topical treatment was renewed.
Stimulated conditions
   The medium was removed and replaced with a fresh culture medium containing the inducer PMA at 0.3 μg/ml and the topical treatment was renewed.
   The skin explants were then incubated for 48 hours.
   At the end of incubation, the explants were washed in a phosphate buffered saline (PBS) solution and 3 punches (4 mm diameter) were performed on each explant and immediately frozen at −80° C.
   All experimental conditions were performed in n=3.

### Differential Expression Analysis

Samples were automatically homogenized using a Precellys/cryolys and the replicates were pooled. Total RNA was extracted from each sample using using NucleoSpin® RNA kit (MachereyNagel)® according to the supplier's instructions.
The amount and quality of total RNA were evaluated for all samples using capillary electrophoresis (Bioanalyzer 2100, Agilent technologies). From each RNA, a labeled and amplified anti-sens RNA (aRNA) was obtained using GeneChip 3′IVT PLUS Kit (Affymetrix). For each labeled and amplified aRNA sample, the profiles were evaluated before and after fragmentation using capillary electrophoresis (Bioanalyzer 2100, Agilent technologies).
Hybridization of fragmented aRNA onto Affymetrix® U219 chip (36,000 transcripts and variants) was performed in the GeneAtlas™ fluidics Affymetrix® hybridization station for 20 hours at 45° C.
U219 chip was analyzed using the GeneAtlas™ Imaging station (Affymetrix®—resolution 2 μm) to generate fluorescence intensity data.

### Results

Detection thresholds in terms of fold change were defined and applied on normalized data; for helpful interpretation, thresholds are defined as followed:

| | Arbitrary classification |
|---|---|
| Fold Change | of observed effects |
| ≥2 | Upregulated probes |
| ≤0.5 | Downregulated probes |

Moreover, probe modulation is considered as significant when its p-value≤0.05.

TABLE 3
Results

| | | | Basal conditions | | Stimulated | |
|---|---|---|---|---|---|---|
| | | | | extract/oil | | extract/oil |
| | Gene | Protein | Oil | (1/49) | Oil | (1/49) |
| Inflammation | CXCL 2 | Macrophage | 0.19 | 0.02 | 0.87 | 0.15 |
| | | inflammatory protein | | | | |
| | | 2-alpha | | | | |
| | CXCL 3 | Macrophage | 0.13 | 0.1 | 0.80 | 0.29 |
| | | inflammatory | | | | |
| | | protein-2-beta | | | | |
| | CXCL 8 | Interleukin 8 | 0.20 | 0.01 | 0.64 | 0.30 |
| | CXCL 10 | Interferon gamma- | 3.46 | 1.06 | 0.84 | 0.82 |
| | | induced protein 10 | | | | |
| | CCL 20 | Chemokin (C-C | 0.15 | 0.04 | 0.72 | 0.57 |
| | | motif) ligand 20 | | | | |
| | IL6 | Interleukin 6 | 0.74 | 0.09 | 1.01 | 0.73 |
| | IL20 | Interleukin 20 | 0.31 | 0.08 | 0.43 | 0.10 |
| | SAA1 | Serum Amyloid A1 | 0.13 | 0.07 | 1.24 | 0.13 |
| | TNFAIP3 | Tumor Necrosis | 0.66 | 0.32 | 1.19 | 0.47 |
| | | Factor, alpha- | | | | |
| | | induced protein 6 | | | | |
| | TNFAIP6 | Tumor Necrosis | 1.06 | 0.16 | 0.83 | 0.33 |
| | | Factor, alpha- | | | | |
| | | induced protein 4 | | | | |
| Peptides Anti | LTF | Lactotransferin | 0.46 | 3.95 | 0.82 | 1.26 |
| Microbien | S100A7 | Psoriasin | 0.69 | 2.61 | 2.18 | 4.63 |
| (PAMs) | S100A7A | Koebnerisin | 0.44 | 6.00 | 1.65 | 2.38 |
| | S100B | S100 calcium | 1.88 | 4.11 | 2.09 | 3.15 |
| | | binding protein B | | | | |
| Oxydative | PTGS2 | Prostaglandin- | 0.49 | 0.08 | 0.54 | 0.30 |
| Stress | | endoperoxide | | | | |
| | | synthase 2 | | | | |
| | GPX3 | Glutathione | 0.37 | 0.28 | 0.79 | 1.01 |
| | | peroxidase 3 | | | | |
| | HMOX1 | Heme oxygenase 1 | 0.21 | 0.07 | 0.12 | 0.69 |
| Keratinocytes | KRT17 | Keratin 17. type I | 0.67 | 0.26 | 0.19 | 0.92 |
| differentiation | KRT6C | Keratin 6C. type II | 0.53 | 2.33 | 1.86 | 4.60 |
| | COL3A1 | Collagen. type III. | 1.20 | 2.89 | 1.04 | 1.23 |
| | | alpha 1 | | | | |
| | SPRR1A | Small proline-rich | 0.88 | 0.38 | 1.30 | 1.38 |
| | | protein 1A | | | | |
| | SPRR2A | Small proline-rich | 0.14 | 0.31 | 0.83 | 2.82 |
| | | protein 2A | | | | |
| | SPRR2B | Small proline-rich | 0.09 | 0.18 | 2.53 | 8.78 |
| | | protein 2B | | | | |
| | SPRR2D | Small proline-rich | 0.30 | 0.28 | 0.67 | 1.31 |
| | | protein 2D | | | | |
| | SPRR2F | Small proline-rich | 0.33 | 0.34 | 0.67 | 1.02 |
| | | protein 2F | | | | |
| | TGM1 | Transglutaminase 1 | 0.79 | 0.30 | 1.56 | 1.87 |
| | LCE 3E | Late Cornified | 0.20 | 0.09 | 2.10 | 1.94 |
| | | Envelope 3E | | | | |
| | CASP14 | Caspase 14 | 0.59 | 0.33 | 2.46 | 1.38 |
| | DSC1 | Desmocollin 1 | 0.82 | 0.72 | 1.28 | 0.99 |
| | CDSN | Corneodesmosin | 0.47 | 0.17 | 0.97 | 1.19 |
| | EREG | Epiregulin | 0.52 | 0.15 | 0.88 | 0.60 |

The tomato peel extract tested diluted in the Tomato seed oil in the proportions 1/49 (w/v) induced significant modulations of the gene expression profile.
Under basal conditions, when compared to the diluent Tomato seed oil, the tomato peel extract tested with the proportions 1/49 (w/v) induced a strong inhibition of genes involved in inflammation (mainly for CXCL8, IL6, CXCL2, in oxidative stress (PTGS2, GPX3, HMOX1), and in terminal keratinocyte differentiation (KRT17, SPRR1A, TGM1, several LCE, EREG, CDSN. In parallel, the tomato peel extract induced an up-regulation of antimicrobial peptides (LTF, S100A7, S100A7A, S100B) and KRT6C and COL3A1 markers.
Under stimulated conditions, the tomato peel extract tested diluted in the tomato seed oil in the proportions 1/49(w/v) induced significant modulations of the gene expression profile. A slight inhibition of genes involved in immune response (CCL22, SERPINB9, CD83, IDO1, IL20, LIF) and involved in matrix degradation (MMP10 and MMP7) were observed under stimulated conditions. The tomato peel extract also induced an increase of markers involved in terminal keratinocyte differentiation (SPRR2B, CASP14, LCE3D, and LCE3E), and a slight up regulation of markers involved in innate immunity (PI3, S100A7, S100B). When compared to the diluent Tomato seed oil, the tomato peel extract induced an inhibition of genes involved in inflammatory response (mainly for CXCL8, IL6, CXCL2, CXCL3, SAA1, IL20, TNFAIP3, and TNFAIP6). In parallel, the tomato peel extract induced an up-regulation of antimicrobial peptide (S100A7A) and markers involved in terminal keratinocyte differentiation (SPRR2B, KRT6C, SPRR2A).

### Conclusion

Under the experimental conditions of this study, the tomato peel extract tested diluted in the tomato seed oil with the proportion 1/49 (w/v), exhibited a clear soothing effect (inhibition of oxidative stress) in this model of ex vivo skin. This extract also clearly promoted the innate immunity of the ex vivo skin through an up-regulation of antimicrobial peptides.

### Example 4—Effects of a Combination of the Tomato Peel Extract According to the Invention and a Tomato Seeds Oil on S100A7 Expression in Reconstructed Human Epidermis

In the present study, the effect of combination of the tomato peel extract and a tomato seeds oil (as prepared in example 2) was investigated in a reconstructed human epidermis (RHE) model by evaluating S100A7 expression using in situ immunolabeling and image analysis.

ABBREVIATIONS

| | |
|---|---|
| AU Arbitrary unit | PMA Phorbol myristate acetate |
| GAM Goat antimouse | RHE Reconstructed human epidermis |
| PBS Phosphate buffered saline | Sd Standard deviation |
| PI Propidium iodide | sem Standard error of the mean |

### Materials and Methods

Biological models
Epidermis: reconstructed human epidermis (RHE), 10-day-old
Culture conditions: 37° C., 5% CO₂
Assay medium: maintenance medium

### Test Samples and Combination (Table 4)

TABLE 4

| Test samples | | Aspect/Storage | Application |
|---|---|---|---|
| Mineral oil | M5904 Sigma | Colorless tick oil | Topical |
| | Aldrich/Merck | Storage: +4° C. | (2.5 |
| | Cas number: | protected from light | μl/RHE) |
| | 8042-47-5 | | |
| Combination of | Tomato peel | Paste | Topical |
| Tomato peel | extract | Storage: +4° C. | (2.5 |
| extract + | | protected from light | μl/RHE) |
| organic seed oil | Organic | Thick homogeneous | |
| (ratio 1/49) | seed oil | turbid liquid | |
| | | Storage: +4° C. | |
| | | protected from light | |
| Combination of | Tomato peel | Paste | Topical |
| Tomato peel | extract | Storage: +4° C. | (2.5 |
| extract + | | protected from light | μl/RHE) |
| organic seed | Organic seed oil | Thick homogeneous | |
| oil + | | turbid liquid | |
| mineral oil | | Storage: +4° C. | |
| (ratio 1/2/47) | | protected from light | |

### Culture and Treatment

At day 10, the RHE were placed in a 12-well plate in assay medium. The RHE were topically treated with the Mineral oil as placebo control (2.5 μl/RHE, ref M5904 Sigma Aldrich/Merck, CAS number 8042-47-5) or the combinations of tested samples (2.5 μl/RHE). All experimental conditions were performed in n=3.

### Paraffin Embedding and Sectioning

The RHE were fixed with formaldehyde solution. Fixed tissues were dehydrated in multiple baths with increasing concentrations of ethanol and then embedded in paraffin. Transversal sections were performed with a microtome (5 μm thickness, 1 slide per RHE) and maintained at room temperature until immunohistolabeling.

### In Situ Immunofluorescent Labeling

The sections were deparaffinized and incubated at 92° C., pH 6, in a retrieval target solution in order to optimize antigen-antibody interaction. The sections were cooled down to room temperature in the same solution. Afterwards, following a PBS-Tween-5% milk saturation, the sections were incubated for 1 hour with the appropriate primary antibody (anti-S100A7). After several washes, the binding sites recognized by the primary antibody were revealed with a secondary fluorescent appropriate antibody (GAM Alexa-488) and the cell nuclei were stained with propidium iodide (PI) solution. The sections were washed in PBS-Tween and mounted in Fluorescent Mounting Medium.

### Microscopic Observation

The sections were observed using a NIKON microscope (objective lens ×40) equipped with a Nikon camera. The images were processed with NIS-Elements software. Five images were captured per replicate.
The fluorescence intensity was measured on the captured images using ImageJ software. The values of fluorescence intensity were normalized to the epidermis area.

### Data Management

Raw data were analyzed using Microsoft Excel® software.
The inter-group comparisons were performed by a non-parametric test of Kruskall-Wallis followed by multiple comparison Dunn's test.
Standard error of the mean: sem=Sd/√n. The standard error of the mean (sem) is a measure of how far the sample mean is likely to be from the true population mean. The sem is calculated as the sd divided by the square root of sample size.

### Results

The results obtained are summarized in Table 5

TABLE 5

| | | S100A7 - Fluroresce intensity/μm² | | | | |
|---|---|---|---|---|---|---|
| | Concentration | Mean (per | | | | P Dunn's multiple |
| | (topical | condition) | Sem | % | Sem | comparison test |
| Test sample | application) | (AU) | (AU) | Control | (%) | Vs control |
| Placebo control | — | 1 | 1 | 100 | 99 | — |
| (mineral oil) | | | | | | |
| P1 = | 2.5 μl/RHE | 23 | 12 | 3190 | 1708 | *** |
| Combination of | | | | | | |
| Tomato peel | | | | | | |
| extract + | | | | | | |
| organic seed oil | | | | | | |
| (ratio 1/49) | | | | | | |
| P2 = | 2.5 μl/RHE | 7 | 2 | 1009 | 312 | * |
| Combination of | | | | | | |
| Tomato peel | | | | | | |
| extract + | | | | | | |
| organic seed | | | | | | |
| oil + | | | | | | |
| mineral oil | | | | | | |
| (ratio 1/2/47) | | | | | | |

Under basal condition, the S100A7 signal was weak and localized in the granular layer of the epidermis.
Under the experimental conditions of the assay, Combination of tomato peel extract according to the invention and organic seed oil (1/49) and Combination of tomato peel extract according to the invention, organic seed oil and mineral oil (1/2/47) tested at 2.5 μl/RHE, induced a significant stimulation of S100A7 expression (respectively 3190%—p<0.001, Dunn's test—and 1009%—p<0.05, Dunn's test—of the control).

### Conclusion

The up-regulation of the gene coding for the antimicrobial peptide (S100A7A) previously observed in example 3 was confirmed as the combination of tomato peel extract according to the invention and organic seed oil (1/49) and the combination of tomato peel extract according to the invention, organic seed oil and mineral oil (1/2/47) increased S100A7 protein expression. We also observed better results with the combination of tomato peel extract according to the invention and organic seed oil (1/49), than the combination of tomato peel extract according to the invention, organic seed oil and mineral oil (1/2/47)

### Example 5—In Vivo Anti-Irritation Assessment

### Goal

To assess the soothing effect of the combination of the tomato peel extract and a tomato seeds oil (as prepared in example 2) after Capsaicin-induced skin irritation in 20 volunteers for 120 minutes.

### Methodology

Twenty volunteers (female), aged between 45 and 65 years old, with visible signs of dry and sensitive skin were included in the study. Four independent experimental areas were defined on the forearms for each volunteer included in the assay. Mild skin irritation was induced on each experimental area by exposure to a Capsaicin occlusive patch for 1 hour. This occlusive patch was prepared and applied by the researcher on four independent experimental areas on the forearms. After patch removal, the tested products were applied by the researcher (3-4 mg/cm2) to the corresponding experimental area. Skin erythema levels were quantified using Mexameter®, at 5 different time points: before start of the treatment (T0), right after inducing a mild skin irritation with Capsaicin occlusive patch (T1 after inducing erythema), as well as 10 min (T2), 30 min (T3), 90 min (T4) and 120 min (T5) after the first application. Changes in erythema levels at each time point were normalized to the status at T1, after inducing irritation. All data were statistically analyzed. Dermatological surveillance was included in the study.

### Sample

The tested products were:
1—Placebo serum formula (water 98%+Phenoxyethanol+sodium stearoyl glutamate+succinoglycan chlorphenesin)
2—Extract according to the invention at concentration 1% (Test 1%) into placebo serum formula
3—Extract according to the invention at concentration 2% (Test 2%) into placebo serum formula

### Results

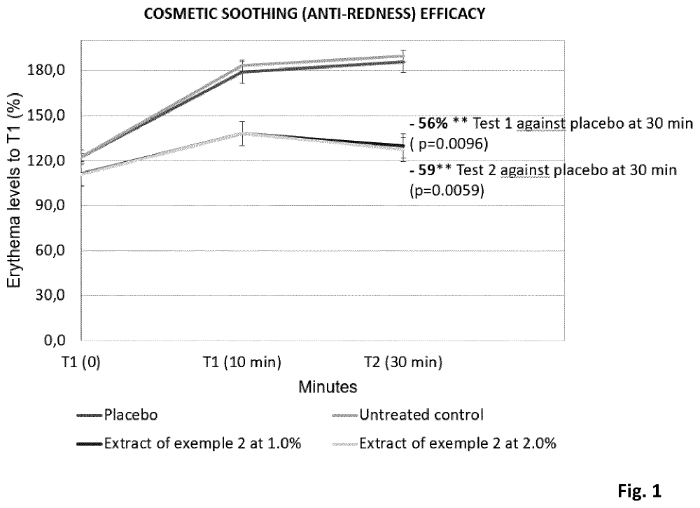

The results are shown in FIG. 1. Results showed treatment with the Capsaicin occlusive patch for 1 hour significantly increased erythema levels in the 4 experimental areas, thus indicating an effective induction of skin irritation.
When the products were compared to the non-treated control after the induction of irritation, results indicated that “Test 2%” decreased erythema levels by 62.11% after 30 minutes of treatment, by 49.55% after 90 min and by 56.53% after 120 min of treatment, while the erythema levels were reduced by 59.52% after 30 min, 52.55% after 90 min and by 75.47% after 120 minutes treatment with “Test 1%”.
In addition, “Test 2%” decreased the skin irritation compared to “Placebo”, evidenced by the reduction of erythema by 58.61% after 30 minutes and, the same way “Test 1%” evidenced a reduction of erythema compared to “Placebo” by 56.01%.
It is worth to note that we did not observe significant differences in erythema levels between the “Placebo” and the non-treated condition at any of the tested time points.
Regarding the skin compatibility and acceptability, none of the volunteers showed any skin acceptability problem or manifested a cutaneous reaction during the treatment.
In conclusion, this clinical study shows that the product “Test 1%” and “Test 2%” have an efficient soothing effect against skin irritation and thus reduce skin irritation.

### Example 6—In Vivo Moisturizing and Barrier Function Capacity Assessment

### Goal

In vivo assessment of the moisturizing and barrier function capacity of 3 treatments (tested products at 0.5% and 1%, and placebo) after topical application in forearms. A kinetic of measurements was established as following: T0 (before the treatment), 30 min, 90 min, 120 min and 24 h after the application.

### Area of Application: Forearm.

### Platform:

30 volunteers (women aged between 40 and 70 years old with dry and sensitive skin condition). The inclusion criteria for sensitive skin volunteers' condition selection were:
Females, 40 to 70 years of age.
Skin phototypes (Fitzpatrick): II, III and IV.
In good general health (physical, mental, and social well-being, not merely the absence of disease/infirmity), according to subject self-report.
Subjects with visible signs of dry and sensitive skin (Subjects were diagnosed as sensitive skin with positive questionnaire and lactic acid test. The questionnaire is positive if it reflects a recent and repeated history of functional symptomatology of skin discomfort by the subject and the lactic acid test in the nasolabial fold is positive if itching is detected in the nasolabial fold after three minutes of application of 1 mL of 10% aqueous lactic acid solution).—Subjects with visible signs of dry and sensitive skin.
   On the other hand, the criteria of exclusion were:
Allergy or reactivity to some of the components of the product, or a product with similar category than tested one.
Relevant cutaneous marks in the experimental areas, which could interfere with the measurements (scars, sunburns, etc.).
In-use relevant pharmacological or hormonal treatment.
Presence of skin diseases or melanomas.

### Samples

The tested products were:
1) Placebo serum formula (=water 98% +Phenoxyethanol+sodium stearoyl glutamate+succinoglycan chlorphenesin) (P.2328)
2) Extract according to the invention at concentration 1% into placebo serum formula (P2330 1%)
3) Extract according to the invention at concentration 0.5% into placebo serum formula (P2329 0.5%)

### Methodology:

A mild skin irritation was induced on each experimental area by erosion by 20 rubbing passes with natural pumice stone by the researcher on four independent experimental areas on the forearms. Before starting of the treatments, we will check for damage to the skin barrier by measuring whether there is a 2-fold increase in TEWL using Tewameter®. After achieving this effect, the tested products were applied by the researcher on the corresponding experimental area. Skin hydration was quantified using Corneometer®, at 5 different time points: After inducing a mild skin irritation with natural pumice stone (T0 after inducing skin irritation), as well as 30 min (T1), 90 min (T2), 120 min (T3) and 24 h (T4) after the first application. After confirmation of a normal distribution of data, results were statistically analyzed applying doubled paired two-way ANOVA statistics test.

### Results

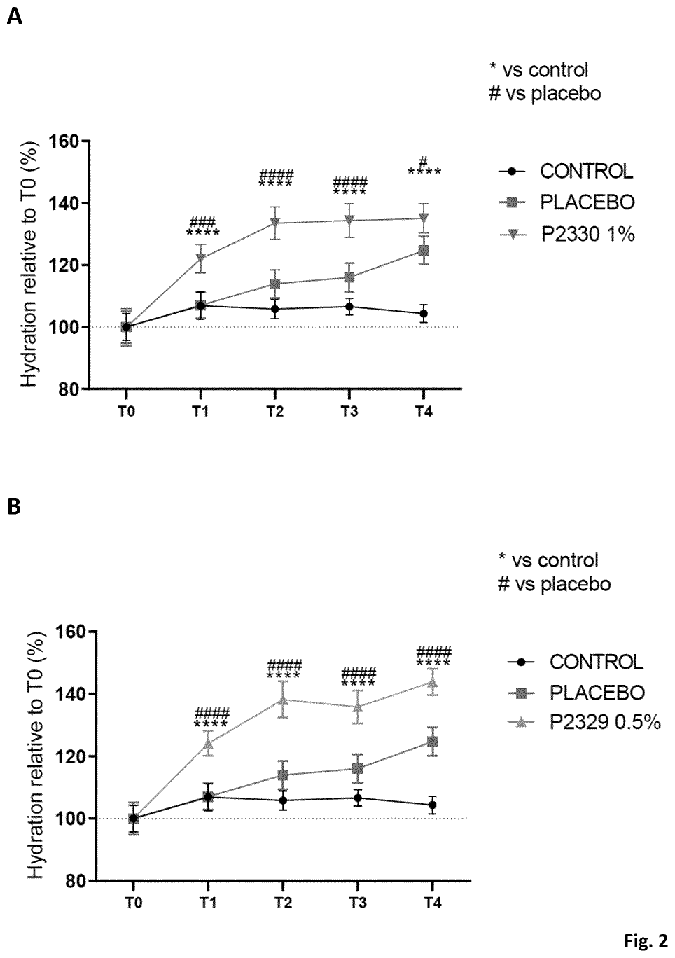

The results for cosmetic hydration effect are shown in FIGS. 2A and B (FIG. 2A for P2330 1%-FIG. 2B for P2329 0.5%). We can observed a significant increase of the skin hydratation after applying the extract according to the invention at concentration 0.5% and 1% into placebo serum formula. Such an increase is not observed with after applying the placebo formula.
Quantification and measurements of the TEWL levels was also carried out using a Tewameter®. The preliminary results show a significant decrease of the TEWL level 90 minutes after the application of the P2330 1% and P2329 0.5%.

### Example 7: Cosmetic and Nutraceutical Products

### Example 7a: Skin Immunity Stimulating Cream for Sensitive Skin

A skin immunity stimulating cream (for sensitive skin) can be prepared by combining the components as described in the following Table 6, the cream comprising the Solanum lycopersicum fruit (Tomato) peel extract of the present invention named Solanum lycopersicum (Tomato) SKIN WAX.

TABLE 6

| | | Content (% |
|---|---|---|
| INCI name | Role | by weight) |
| Aqua | Solvent | QSP |
| Solanum lycopersicum | Active ingredient | 25 |
| (Tomato) SKIN WAX:tomato | (Skin Immunity Boost | |
| seeds oil (1:49) | Agent): emollient | |
| Ammonium polyacryldimethyl | Gelling agent in | 1.5 |
| tauramide/Xanthan gum | aqueous phase | |
| Glycerin | Mooring | 5 |
| Co-emulsifier | Co-emulsifier | 2 |
| Dextrin palmitate/ethylhexanoate | Gelling agent in | 1 |
| | the oily phase | |
| Phenoxyethanol | Conservative | 0.5 |

### Example 7b: High Performance Cleansing Balm

A high-performance cleansing balm can be prepared by combining the components as described in the following Table 7, the balm comprising Solanum lycopersicum fruit (Tomato) peel extract of the present invention named Solanum lycopersicum (Tomato) SKIN WAX.

TABLE 7

| | | Content (% |
|---|---|---|
| INCI name | Role | by weight) |
| Aqua | Solvent | QSP 100 |
| Solanum lycopersicum (Tomato) | Active ingredient | 1 |
| SKIN WAX:tomato seeds oil | (Skin Immunity | |
| (weight ratio dilution of 1:2) | Boost Agent) | |
| Sodium hydroxyde | Based | 1-3 |
| Panthenol | Active | 1-3 |
| Bis-PEG-15 methyl ether dimethicone | Surfactant | 1-3 |
| Dipropylene glycol | Solvent | 1-3 |
| Mannitol, xylitol, rhamnose, | Assets | 1-3 |
| fructooligosaccharides | | |
| Ammonium acryloyldimethyltaurate/VP | Thickening | 0.3-1.5 |
| copolymer | | |
| Hydroxyethylcellulose | Thickening | 0.3-15 |
| Propylene glycol | Solvent | 0.3-2 |
| Biosaccharide gum-1 | Conditioner | 0.3-0.6 |
| Phenoxyethanol | Conservative | 0.2-0.4 |

### Example 7c: A Cream for Prevention or Treatment of Atopic Skin Disorders

A cream for prevention or treatment of atopic skin disorders can be prepared by combining the components as described in the following Table 8, the cream comprising Solanum lycopersicum fruit (Tomato) peel extract of the present invention named Solanum lycopersicum (Tomato) SKIN WAX.

TABLE 8

| | | Content (% |
|---|---|---|
| Inci names | Role | by weight) |
| Solanum lycopersicum (Tomato) | Active ingredient | 0.5 to 30% |
| SKIN WAX:tomato seeds oil | (Skin Immunity | |
| (weight ratio dilution of 1:49) | Boost Agent): | |
| | Emollient | |
| Butylene glycol | Skin conditioning | 3.0 |
| Glycerin | Mooring | 5.0 |
| PEG-60 Hydrogenated Castor Oil | Surfactant | 0.2 |
| Ethanol | Solvent | 8.0 |
| Citric acid | Buffering | 0.02 |
| Sodium citrate | Buffering | 0.06 |
| ACQUA | | QSP 100 |

### Example 7d: Cleansing Foam for Face Wash

An atopy skin cleansing agent (cleansing foam for face wash) comprising Solanum lycopersicum fruit (Tomato) peel extract of the present invention named Solanum lycopersicum (Tomato) SKIN WAX, is formulated by combining the components as described in the following

Table 9.

TABLE 9

| | | CONTENT (% |
|---|---|---|
| INCI NAMES | ROLE | by weight) |
| Solanum lycopersicum (Tomato) | Active ingredient | 0.5 to 30% |
| SKIN WAX:tomato seeds oil | (Skin Immunity | |
| (weight ratio dilution of 1:49) | Boost Agent): | |
| | Emollient | |
| Sodium Stearoyl Glutamate | Cleansing | 20.0 |
| Glycerin | Mooring | 10.0 |
| PEG-400 | Emulsion stabilising | 15.0 |
| Propylene glycol | Skin conditioning | 10.0 |
| Oleyl Alcohol | Viscosity controlling | 3.0 |
| Trilaurin | Skin conditioning | 2.0 |
| Phenoxyethanol | preservative | 0.2 |
| ACQUA | | QSP 100 |

### Example 7e: Immunity Boost After Sun Serum for Sensitive Skin

Immunity Boost Serum (For Oily Skin) comprising Solanum lycopersicum fruit (Tomato) peel extract of the present invention named Solanum lycopersicum (Tomato) SKIN WAX of according to the present invention, is formulated by combining the components as described in the following Table 10.

TABLE 10

| | | CONTENT |
|---|---|---|
| | | (% by |
| INCI name | Role | weight) |
| Aqua | Solvent | QSP 100 |
| Solanum lycopersicum | Active ingredient | 0.5-4% |
| (Tomato) SKIN WAX | (Skin Immunity | |
| | Boost Agent) | |
| Sodium hydroxyde | Based | 1-3 |
| Vegetable/mineral oil | emmollient | 10-20 |
| Panthenol | Active | 1-3 |
| Bis-PEG-15 methyl ether dimethicone | Surfactant | 1-3 |
| Dipropylene glycol | Solvent | 1-3 |
| Mannitol, xylitol, rhamnose, | Assets | 1-3 |
| fructooligosaccharides | | |
| Ammonium acryloyldimethyltaurate/VP | Thickening | 0.3-1.5 |
| copolymer | | |
| Hydroxyethylcellulose | Thickening | 0.3-15 |
| Propylene glycol | Solvent | 0.3-2 |
| Biosaccharide gum-1 | Conditioner | 0.3-0.6 |
| Phenoxyethanol | Conservative | 0.2-0.4 |

### Example 7f: Tomato′ Immunity Lipstick Formula

Tomato′ Immunity Lipstick Formula comprising Solanum lycopersicum fruit (Tomato) peel extract of the present invention named Solanum lycopersicum (Tomato) SKIN WAX is formulated by combining the components as described in the following Table 11.

TABLE 11

| | | Quantity |
|---|---|---|
| INCI name | Role | (%) |
| Solanum lycopersicum (Tomato) | Active ingredient | 5-10% |
| SKIN WAX:tomato seeds oil | (Skin Immunity | |
| (1/49) | Boost Agent): | |
| | Emollient | |
| CERA ALBAN | Texturizing agent | 90-95% |

### Example 7g: Oral Taking Capsules for Food Supplement Or Nutricosmetic Supplement to Boost Immunity

ORAL TAKING CAPSULES Formula comprising Solanum lycopersicum fruit (Tomato) peel extract of the present invention named Solanum lycopersicum (Tomato) SKIN WAX is formulated by combining the components as described in the following Table 12.

TABLE 12

| | Capsule | Quantity |
|---|---|---|
| INCI name | weight | (%) |
| Solanum lycopersicum (Tomato) | 250 mg | 100% |
| SKIN WAX and vegetable oil/mineral | | |
| oil (weight ratio 1:49) | | |

### Example 7h : Shampoo for Prevent or Treating Dandruff (for Sensitive Scalp)

Shampoo for prevent or treating dandruff formula comprising Solanum lycopersicum fruit (Tomato) peel extract of the present invention named Solanum lycopersicum (Tomato) SKIN WAX is formulated by combining the components as described in the following Table 13.

TABLE 13

| | | |
|---|---|---|
| | | Content |
| | | (% by |
| INCI name | Role | weight) |

Phase A

| | | |
|---|---|---|
| Solanum lycopersicum (Tomato) | Active ingredient | 1-2 |
| SKIN WAX | (Hair conditioning | |
| | agent) | |
| Pantenol | Hair conditioning | 0.3-0.5 |
| | agent | |
| Polyquaternium | Conditioning agent | 0.5-1 |
| Citric acid | Buffering | 0.7 |

Phase B

| | | |
|---|---|---|
| DISODIUM LAURETH | Foaming | 8-12 |
| SULFOSUCCINATE | | |
| HYDROXYPROPYLTRIMONIUM | Hair conditioning | 6-10 |
| HYDROLYZED WHEAT PROTEIN | | |
| ZINC COCETH SULFATE | Surfactant | 20-30 |
| OCOGLUCOSIDES | Surfactant | 3-6 |
| HYDROXYPROPYLTRIMONIUM | | |
| CHLORIDE | | |

Phase C

| | | |
|---|---|---|
| SODIUM LAUROYL SARCOSINATE | Cleansing | 4-6 |
| PIROCTONE OLAMINE | Preservative | 0.5 |

Phase D

| | | |
|---|---|---|
| POTASSIUM COCOYL GLYCINATE | Surfactant | 1 |

Phase E

| | | |
|---|---|---|
| PEG-90 GLYCERYL ISOSTEARATE | Cleansing | 2-3 |

Phase F

| | | |
|---|---|---|
| Parfum | Perfuming | QS |
| PEG-120 METHYL GLUCOSE | Emulsifying | 1 |
| DIOLEATE | | |
| ACQUA | | QSP 100 |

1-20. (canceled)

21. A cosmetic method for maintaining, balancing or reinforcing the natural defenses of the skin or mucous membranes, comprising the oral or topical administration to a subject of a lipophilic Solanum lycopersicum fruit peel extract comprising at most 0.5% by weight of lycopene, at least 5% by weight of amyrins and at least 1.5% by weight of sterols, in relation to the total weight of the extract.

22. The cosmetic method of claim 21, wherein the oral or topical administration of the lipophilic Solanum lycopersicum fruit peel extract reinforces skin barrier function, and/or maintains or increases the resistance or the tolerance of the skin against an external aggression or stress, and/or prevents or treats a non-pathological alteration of the skin caused by an external aggression or stress, and/or improves or restores skin comfort and/or restores and/or maintains a healthy skin microbiome.

23. The cosmetic method of claim 21, wherein the oral or topical administration of the lipophilic Solanum lycopersicum fruit peel extract soothes the skin.

24. The cosmetic method of claim 21, wherein the oral or topical administration of the lipophilic Solanum lycopersicum fruit peel extract induces the expression of antimicrobial peptides (AMPs).

25. The cosmetic method of claim 21, wherein the lipophilic Solanum lycopersicum fruit peel extract is administered in combination with a vegetable or mineral oil, and/or with Solanum lycopersicum seed oil.

26. The cosmetic method of claim 25, wherein the weight ratio of the lipophilic Solanum lycopersicum fruit peel extract to the vegetal or mineral oil is from 1:2 to 1:70.

27. The cosmetic method of claim 25, wherein the vegetal oil and the lipophilic Solanum lycopersicum fruit peel extract are obtained by simultaneous or separate extraction(s) of tomato byproducts.

28. The cosmetic method of claim 21, wherein the lipophilic Solanum lycopersicum fruit peel extract comprises at most 0.3% by weight of lycopene, in relation to the total weight of the extract.

29. The cosmetic method of claim 28, wherein the lipophilic Solanum lycopersicum fruit peel extract comprises at most at most 0.1% by weight, of lycopene, in relation to the total weight of the extract.

30. The cosmetic method of claim 21, wherein the amyrins are α-amyrin, β-amyrin and/or δ-amyrin.

31. The cosmetic method of claim 21, wherein the lipophilic Solanum lycopersicum fruit peel extract comprises at least 1.5% by weight of α-amyrin, at least 1% by weight of β-amyrin and at least 2.5% by weight of δ-amyrin, in relation to the total weight of the extract.

32. The cosmetic method of claim 21, wherein the lipophilic Solanum lycopersicum fruit peel extract comprises at least 1% by weight of β-sitosterol and stigmasterol, in relation to the total weight of the extract.

33. The cosmetic method of claim 21, said method comprising the topical administration to a subject of a cosmetic composition comprising the lipophilic Solanum lycopersicum fruit peel extract in a weight content from 0.01% to 30%, in relation to the total weight of the composition, said composition comprising at least one cosmetically acceptable excipient.

34. The cosmetic method of claim 21, wherein the subject is a healthy subject having sensitive skin, dry skin, aged-skin, blemish-prone skin, acne-prone skin, atopic-prone skin or skin weakened by an external insult or stress.

35. A method for treating a skin disease characterized by an altered or unbalanced innate immune response comprising the oral or topical administration to a subject in need thereof of a composition comprising a lipophilic Solanum lycopersicum fruit peel extract comprising at most 0.5% by weight of lycopene, at least 5% by weight of amyrins and at least 1.5% by weight of sterols, in relation to the total weight of the extract, as an active ingredient.

36. The method of claim 35, wherein the skin disease is eczema, psoriasis, rosacea, acne, dermatitis, atopic dermatitis, and irritated skin.

37. A lipophilic Solanum lycopersicum fruit peel extract comprising:
at most 0.3% by weight of lycopene, in relation to the total weight of the extract;
at least 5% by weight of amyrins in relation to the total weight of the extract;
at least 1.5% by weight of sterols, in relation to the total weight of the extract.

38. A cosmetic composition comprising a lipophilic Solanum lycopersicum fruit peel extract according to claim 37, in a weight content from 0.01% to 30% in relation to the total weight of the composition, and at least one cosmetic acceptable excipient.
